(19) United States
(12) Patent Application Publication
(10) Pub. No.: US 2022/0193081 A1
(43) Pub. Date: Jun. 23, 2022

(54) LIQUID SILDENAFIL CITRATE COMPOSITIONS

(71) Applicant: Ellamed IP & Services PTE. LTD., Singapore (SG)

(72) Inventor: Moshe Rogosnitzky, Telz Stone (IL)

(73) Assignee: Ellamed IP & Services PTE. LTD., Singapore (SG)

(21) Appl. No.: 17/604,618

(22) PCT Filed: Apr. 17, 2020
(86) PCT No.: PCT/IB2020/053660

§ 371 Date: Oct. 18, 2021

Related U.S. Application Data
(60) Provisional application No. 62/835,523, filed on Apr. 18, 2019.

(51) Int. Cl. A61K 31/519; A61K 9/08; A61K 47/10; A61K 47/12

(52) U.S. Cl. CPC A61K 31/519; A61K 47/12; A61K 47/10; A61K 9/08

(57) ABSTRACT

The invention is directed to liquid pharmaceutical compositions comprising at least 15mg/ml of sildenafil citrate, their use in treating erectile dysfunction and their methods of manufacture.

### BACKGROUND

Sildenafil citrate is an FDA and EMEA approved drug for treatment of erectile dysfunction and pulmonary arterial hypertension (PAH).

There are reports that sildenafil citrate is being used for off-label indications including, but not limited to, prevention of high-altitude pulmonary edema associated with altitude sickness, treating lung fibrosis, primary pulmonary hypertension, secondary pulmonary hypertension, hypoxia induced pulmonary hypertension, neonatal pulmonary hypertension, pediatric pulmonary hypertension, nonoperable chronic thromboembolic pulmonary hypertension, severe coronary artery disease, age-related macular degeneration, brachial artery flow-mediated dilatation (in type 2 diabetes), Raynaud's syndrome, anal fissures, postmenopausal female sexual dysfunction, female sexual arousal disorder, digital ulcers secondary to systemic sclerosis, migraine, premature ejaculation, sickle-cell disease with pulmonary hypertension, achalasia (esophageal motility dysfunction), severe digital ischemia, recurrent ischemic priapism, severe lymphatic formation, congestive heart failure, diastolic dysfunction, tunical fibrosis, multiple sclerosis, intrauterine growth restriction, chronic pelvic pain, Alzheimer's disease, stroke, preeclampsia, gastroparesis, glucose dyscontrol in diabetes, primary dysmenorrheal pain, for increasing exercise capacity during hypoxia, increasing uterine artery blood flow and endometrial thickness to promote in-vitro fertilization (IVF). Sildenafil citrate has also been proposed as a treatment for prostate cancer, pancreatic cancer, ovarian cancer, stomach cancer, obesity, Crohn's disease, spastic esophageal disorder, reduction of alcohol induced gastric damage and other conditions.

Sildenafil citrate is commonly marketed as VIAGRA® (for treatment of erectile dysfunction) and REVATIO® (for treatment of pulmonary hypertension), both manufactured by Pfizer Pharmaceuticals. Generic versions of sildenafil citrate are also available.

VIAGRA® is commonly supplied as 25, 50 or 100 mg tablets and is to be taken not more than once per day 0.5 to 4 hours prior to intercourse. REVATIO® is most often supplied as 20 mg tablets to be taken 3 times daily. REVATIO® is also available in injectable form as a clear colourless, sterile, ready to use solution containing 10 mg of sildenafil citrate per 12.5 ml of solution or as a powder for oral suspension at a concentration of 10 mg/ml. Each ml of the solution product contains 1.124 mg sildenafil citrate, 50.5 mg dextrose and water for injection. The injectable form of REVATIO® is most often administered intravenously, typically in a hospital setting. Additional ingredients in the REVATIO® powder product include colloidal silicon dioxide, sucralose, sorbitol, sodium benzoate, sodium citrate, flavourings and xanthan gum. The REVATIO® powder product has a slower absorption rate than would be expected for a solution with a similar concentration. In addition, the presence of some of the additional ingredients makes this product difficult to tolerate for people with known sensitivities to the additional excipients.

Whether provided as tablets or oral suspension, sildenafil citrate exhibits an absolute bioavailability of about 41% and is reported to result in maximum observed plasma concentrations within 30 to 120 minutes following oral dosing in a fasted state. The rate of absorption is reportedly reduced if taken with a high fat meal.

According to the US package insert for VIAGRA®, the solubility of sildenafil citrate in water is 3.5 mg/ml. The EMEA CHMP Assessment Report for VIZARSIN® (International Nonproprietary Name: sildenafil) indicates that it is insoluble in ethanol, chloroform and acetone, but soluble in methanol and dimethyl sulfoxide (DMSO). The Jordanian Pharmaceutical Manufacturing Co. reports that sildenafil citrate is about 3.5 times less soluble in ethanol than in water (˜1 mg/ml). The low water solubility of sildenafil citrate and/or its high pre-systemic elimination each independently contribute to its low oral bioavailability.

WO2015140748 (Rogosnitzky) describes the preparation of liquid oral dosage forms of sildenafil citrate at a concentration of at least 7 mg/ml in water, at least 20% alcohol and with a pH in the range of 4.4 to 4.55. The liquid dosage forms are each prepared at temperatures above at least 70° C. It is also shown that such liquid dosage forms have a shorter onset of action than the marketed VIAGRA® tablets.

WO0135926 (Vallabhaneni) describes the preparation of liquid nasal dosage forms of up to 10% sildenafil citrate in alcohols at a pH adjusted to no more than 4.0 which are claimed to have a shorter onset of action than the marketed VIAGRA® tablets. Said dosage forms can be administered as single dose sprays of up to 15 mg sildenafil citrate.

WO2013085904 (Bergstrom) describes the preparation of liquid oral spray dosage forms of up to 12% sildenafil citrate in a co-solvent mix of water, propylene glycol and ethanol at a pH of about 1.5 and less than 3.0. Said dosage forms can be administered as single dose sprays of up to 25 mg sildenafil citrate.

Despite these disclosures, there remains the need for stable liquid pharmaceutical compositions of sildenafil citrate which are available for administration of larger doses of sildenafil citrate in small volumes and which also have a simplified and safer method of manufacturing.

### SUMMARY OF THE INVENTION

The present invention relates to liquid pharmaceutical compositions comprising at least 15 mg/ml of sildenafil citrate and having a pH of between about 5.5 and about 5.7, their use in treating erectile dysfunction and their methods of manufacture.

### DETAILED DESCRIPTION OF THE INVENTION

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” refers to a value of 7.2 to 8.8, inclusive; as another example, the phrase “about 8%” refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1 to 2”, “1 to 2 and 4 to 5”, “1 to 3 and 5”, and the like. In addition, when a list of alternatives is positively provided, such a listing can also include embodiments where any of the alternatives may be excluded. For example, when a range of “1 to 5” is described, such a description can support situations whereby any of 1, 2, 3, 4, or 5 are excluded; thus, a recitation of “1 to 5” may support “1 and 3-5, but not 2”, or simply “wherein 2 is not included.”

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a human subject induces a desired pharmacological and/or physiologic effect by local and/or systemic action.

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder.

As employed above and throughout the disclosure the term “effective amount” refers to an amount effective, at dosages, and for periods of time necessary, to achieve the desired result with respect to the treatment of the relevant disorder, condition, or side effect. It will be appreciated that the effective amount of components of the present invention will vary from patient to patient not only with respect to the particular compound, component or composition selected, the route of administration, and the ability of the components to elicit a desired result in the individual, but also with respect to factors such as the disease state or severity of the condition to be alleviated, hormone levels, age, sex, weight of the individual, the state of being of the patient, and the severity of the pathological condition being treated, concurrent medication or special diets then being followed by the particular patient, and other factors which those skilled in the art will recognize, with the appropriate dosage being at the discretion of the attending physician. Dosage regimes may be adjusted to provide improved therapeutic response. An effective amount is also one in which any toxic or detrimental effects of the components are outweighed by the therapeutically beneficial effects.

The present invention relates to liquid pharmaceutical compositions comprising at least 10 mg/ml of sildenafil citrate and having a pH of between about 5.2 and about 5.8.

As used herein, the term csildenafil citrate' refers to the compound 1-[[3-(4,7-Dihydro-1-methyl-7-oxo-3-propyl-1H-pyrazolo[4,3-d]pyrimidin-5-yl)-4-ethoxyphenyl]sulfonyl]-4-methylpiperazine citrate salt, hydrates and solvates thereof.

As used herein, the term csildenafil Impurity B′ refers to the compound 5-[2-Ethoxy-5-[(4-methyl-4-oxido-1-piperazinyl)sulfonyl]phenyl]-1,6-dihydro-1-methyl-3-propyl-7H-pyrazolo[4,3-d]pyrimidin-7-one.

As used herein, the term ‘sildenafil Impurity D’ refers to the compound 3-(4,7-Dihydro-1-methyl-7-oxo-3-propyl-1H-pyrazolo[4,3-d]pyrimidin-5-yl)-4-ethoxybenzenesulfonic acid.

As used herein, the term ‘hydrate’ refers to sildenafil citrate having a stoichiometric or non-stoichiometric amount of water bound by non-covalent intermolecular forces, for example, the hydrated form of sildenafil hemi-citrate. The hydrate may comprise at least one equivalent of water, for example, one to five equivalents of water. It may be prepared by crystallizing the compounds, or pharmaceutically acceptable salt thereof, in water or an aqueous solvent.

As used herein, the term ‘solvate’ refers to sildenafil citrate having a stoichiometric or non-stoichiometric amount of a solvent bound by non-covalent intermolecular forces. In a preferred embodiment, the solvent is non-volatile, non-toxic, and suitable for administration to humans including, for example, ethanol, methanol, propanol, and methylene chloride.

In one embodiment of the invention, the liquid pharmaceutical composition comprises at least 10 mg/ml of sildenafil citrate. In another embodiment of the invention, the liquid pharmaceutical composition comprises at least 15 mg/ml, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145 or at least 150 mg/ml of sildenafil citrate. In a preferred embodiment of the invention, the liquid pharmaceutical composition comprises at least 15 mg/ml. In another preferred embodiment of the invention, the liquid pharmaceutical composition comprises at least 20 mg/ml. In another preferred embodiment of the invention, the liquid pharmaceutical composition comprises at least 25 mg/ml. In another preferred embodiment of the invention, the liquid pharmaceutical composition comprises at least 50 mg/ml. In another preferred embodiment of the invention, the liquid pharmaceutical composition comprises at least 70 mg/ml. In another preferred embodiment of the invention, the liquid pharmaceutical composition comprises at least 80 mg/ml.

In one embodiment of the invention, the liquid pharmaceutical composition comprises about 10 mg/ml of sildenafil citrate. In another embodiment of the invention, the liquid pharmaceutical composition comprises about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145 or about 150 mg/ml. In one preferred embodiment of the invention, the liquid pharmaceutical composition comprises about 15 mg/ml. In another preferred embodiment of the invention, the liquid pharmaceutical composition comprises about 20 mg/ml. In another preferred embodiment of the invention, the liquid pharmaceutical composition comprises about 25 mg/ml.

In one embodiment of the invention, the liquid pharmaceutical composition comprises between about 15 and about 150 mg/ml of sildenafil citrate. In another embodiment of the invention, the liquid pharmaceutical composition comprises between about 15 and about 120 mg/ml, between about 15 and about 100 mg/ml, between about 15 and about 80 mg/ml, between about 15 and about 50 mg/ml, between about 15 and about 40 mg/ml, between about 15 and about 30 mg/ml, between about 15 and about 25 mg/ml, between about 20 and about 150 mg/ml, between about 20 and about 120 mg/ml, between about 20 and about 100 mg/ml, between about 20 and about 80 mg/ml, between about 20 and about 50 mg/ml, between about 20 and about 40 mg/ml, between about 20 and about 30 mg/ml, between about 20 and about 25 mg/ml, between about 25 and about 150 mg/ml, between about 25 and about 120 mg/ml, between about 25 and about 100 mg/ml, between about 25 and about 80 mg/ml, between about 25 and about 50 mg/ml, between about 25 and about 40 mg/ml or between about 25 and about 30 mg/ml.

In one embodiment of the invention, the liquid pharmaceutical composition has a pH of between about 5.2 and about 5.8. In a preferred embodiment of the invention, the liquid pharmaceutical composition has a pH of between about 5.4 and about 5.8. In a more preferred embodiment of the invention, the liquid pharmaceutical composition has a pH of between about 5.5 and about 5.7.

In one embodiment of the invention, the liquid pharmaceutical composition has a pH of about 5.2. In another embodiment of the invention, the liquid pharmaceutical composition has a pH of about 5.2, about 5.3, about 5.4, about 5.5, about 5.6, about 5.7 or about 5.8. In a preferred embodiment of the invention, the liquid pharmaceutical composition has a pH of about 5.5. In another preferred embodiment of the invention, the liquid pharmaceutical composition has a pH of about 5.6. In another preferred embodiment of the invention, the liquid pharmaceutical composition has a pH of about 5.7.

In one embodiment of the invention, the liquid pharmaceutical composition comprises at least 10, at least 15, at least 20 or at least 25 mg/ml of sildenafil citrate and has a pH of between about 5.5 and about 5.7. In another embodiment of the invention, the liquid pharmaceutical composition comprises about 10, about 15, about 20 or about 25 mg/ml of sildenafil citrate and has a pH of between about 5.5 and about 5.7. In another embodiment of the invention, the liquid pharmaceutical composition comprises between about 15 and about 40, between about 20 and about 30 or between about 20 and about 25 mg/ml of sildenafil citrate and has a pH of between about 5.5 and about 5.7.

In one embodiment of the invention, the liquid pharmaceutical composition comprises at least 15, at least 20 or at least 25 mg/ml of sildenafil citrate and has a pH of about 5.5. In another embodiment of the invention, the liquid pharmaceutical composition comprises about 15, about 20 or about 25 mg/ml of sildenafil citrate and has a pH of about 5.5.

In one embodiment of the invention, the liquid pharmaceutical composition comprises at least 15, at least 20 or at least 25 mg/ml of sildenafil citrate and has a pH of about 5.6. In another embodiment of the invention, the liquid pharmaceutical composition comprises about 15, about 20 or about 25 mg/ml of sildenafil citrate and has a pH of about 5.6.

In one embodiment of the invention, the liquid pharmaceutical composition comprises at least 15, at least 20 or at least 25 mg/ml of sildenafil citrate and has a pH of about 5.7. In another embodiment of the invention, the liquid pharmaceutical composition comprises about 15, about 20 or about 25 mg/ml of sildenafil citrate and has a pH of about 5.7.

The present invention also relates to liquid pharmaceutical compositions comprising at least 15 mg/ml of sildenafil citrate and between about 40% and about 60% aqueous ethanol and having a pH of between about 5.5 and about 5.7.

As used herein, the term ‘aqueous ethanol’ shall refer to solutions of ethanol dissolved in water. The amount of ethanol is such a solution is typically described as the percentage volume of ethanol to water in the mixture (v/v). Thus, 100 ml of 40% aqueous ethanol would be understood to refer to a 100 ml mixture containing 40 ml ethanol dissolved in 60 ml water.

In one embodiment of the invention, the liquid pharmaceutical compositions of the invention comprise between about 40% and about 60% aqueous ethanol.

In one embodiment of the invention, the liquid pharmaceutical compositions of the invention comprise about 40% aqueous ethanol. In another embodiment of the invention, the liquid pharmaceutical compositions of the invention comprise about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59% or about 60% aqueous ethanol. In a preferred embodiment of the invention, the liquid pharmaceutical compositions of the invention comprise about 50% aqueous ethanol.

In one embodiment of the invention, the liquid pharmaceutical composition of the invention comprises at least 10 mg/ml of sildenafil citrate and between about 40% and about 60% aqueous ethanol and has a pH of between about 5.2 and about 5.8.

In one embodiment of the invention, the liquid pharmaceutical composition comprises at least 10, at least 15, at least 20 or at least 25 mg/ml of sildenafil citrate and between about 40% and about 60% aqueous ethanol and has a pH of between about 5.5 and about 5.7. In another embodiment of the invention, the liquid pharmaceutical composition comprises about 15, about 20 or about 25 mg/ml of sildenafil citrate and between about 40% and about 60% aqueous ethanol and has a pH of between about 5.5 and about 5.7.

In one embodiment of the invention, the liquid pharmaceutical composition comprises at least 15, at least 20 or at least 25 mg/ml of sildenafil citrate and between about 40% and about 60% aqueous ethanol and has a pH of about 5.5. In another embodiment of the invention, the liquid pharmaceutical composition comprises about 15, about 20 or about 25 mg/ml of sildenafil citrate and between about 40% and about 60% aqueous ethanol and has a pH of about 5.5.

In one embodiment of the invention, the liquid pharmaceutical composition comprises at least 15, at least 20 or at least 25 mg/ml of sildenafil citrate and between about 40% and about 60% aqueous ethanol and has a pH of about 5.6. In another embodiment of the invention, the liquid pharmaceutical composition comprises about 15, about 20 or about 25 mg/ml of sildenafil citrate and between about 40% and about 60% aqueous ethanol and has a pH of about 5.6.

In one embodiment of the invention, the liquid pharmaceutical composition comprises at least 15, at least 20 or at least 25 mg/ml of sildenafil citrate and between about 40% and about 60% aqueous ethanol and has a pH of about 5.7. In another embodiment of the invention, the liquid pharmaceutical composition comprises about 15, about 20 or about 25 mg/ml of sildenafil citrate and between about 40% and about 60% aqueous ethanol and has a pH of about 5.7.

In one embodiment of the invention, the liquid pharmaceutical composition comprises at least 10, at least 15, at least 20 or at least 25 mg/ml of sildenafil citrate and about 50% aqueous ethanol and has a pH of between about 5.5 and about 5.7. In another embodiment of the invention, the liquid pharmaceutical composition comprises about 15, about 20 or about 25 mg/ml of sildenafil citrate and about 50% aqueous ethanol and has a pH of between about 5.5 and about 5.7. In another embodiment of the invention, the liquid pharmaceutical composition comprises between about 15 and about 40, between about 20 and about 30 or between about 20 and about 25 mg/ml of sildenafil citrate and about 50% aqueous ethanol and has a pH of between about 5.5 and about 5.7.

In one embodiment of the invention, the liquid pharmaceutical composition comprises at least 15, at least 20 or at least 25 mg/ml of sildenafil citrate and about 50% aqueous ethanol and has a pH of about 5.5. In another embodiment of the invention, the liquid pharmaceutical composition comprises about 15, about 20 or about 25 mg/ml of sildenafil citrate and about 50% aqueous ethanol and has a pH of about 5.5.

In one embodiment of the invention, the liquid pharmaceutical composition comprises at least 15, at least 20 or at least 25 mg/ml of sildenafil citrate and about 50% aqueous ethanol and has a pH of about 5.6. In another embodiment of the invention, the liquid pharmaceutical composition comprises about 15, about 20 or about 25 mg/ml of sildenafil citrate and about 50% aqueous ethanol and has a pH of about 5.6.

In one embodiment of the invention, the liquid pharmaceutical composition comprises at least 15, at least 20 or at least 25 mg/ml of sildenafil citrate and about 50% aqueous ethanol and has a pH of about 5.7. In another embodiment of the invention, the liquid pharmaceutical composition comprises about 15, about 20 or about 25 mg/ml of sildenafil citrate and about 50% aqueous ethanol and has a pH of about 5.7.

The present invention also relates to liquid pharmaceutical compositions comprising at least 15 mg/ml of sildenafil citrate further comprising sucralose.

As used herein, the term ‘sucralose’ refers to the compound 1,6-Dichloro-1,6-dideoxyl3-D-fructofuranosyl-4-chloro-4-deoxy-α-D-galactopyranoside.

In one embodiment of the invention, the concentration of sucralose in the liquid pharmaceutical compositions of the invention shall match the concentration of sildenafil citrate in said compositions. Thus, a liquid pharmaceutical composition comprising at least 15 mg/ml of sildenafil citrate and sucralose shall be understood to refer to a liquid composition comprising at least 15 mg/ml of sildenafil citrate and at least 15 mg/ml of sucralose. In another embodiment of the invention, the concentration of sucralose in the liquid pharmaceutical composition of the invention shall be different from the concentration of sildenafil citrate in said compositions. In one embodiment of the invention, the concentration of sucralose in the liquid pharmaceutical composition of the invention comprises between about 1 and about 20 mg/ml, for example about 1, about 2, about 3, about 4, about 5, about 6, about 7, about 8, about 9, about 10, about 11, about 12 about 13, about 14, about 15, about 16, about 17, about 18, about 19 or about 20 mg/ml.

In one embodiment of the invention, the liquid pharmaceutical composition comprises at least 10 mg/ml of sildenafil citrate and sucralose and has a pH of between about 5.5 and about 5.7. In one embodiment of the invention, the liquid pharmaceutical composition comprises at least 10, at least 15, at least 20 or at least 25 mg/ml of sildenafil citrate and sucralose and between about 40% and about 60% aqueous ethanol and has a pH of between about 5.5 and about 5.7. In another embodiment of the invention, the liquid pharmaceutical composition comprises about 15, about 20 or about 25 mg/ml of sildenafil citrate and sucralose and between about 40% and about 60% aqueous ethanol and has a pH of between about 5.5 and about 5.7.

In one embodiment of the invention, the liquid pharmaceutical composition comprises at least 10, at least 15, at least 20 or at least 25 mg/ml of sildenafil citrate, between about 1 and about 20 mg/ml of sucralose and between about 40% and about 60% aqueous ethanol and has a pH of between about 5.5 and about 5.7. In another embodiment of the invention, the liquid pharmaceutical composition comprises about 15, about 20 or about 25 mg/ml of sildenafil citrate, between about 1 and about 20 mg/ml of sucralose and between about 40% and about 60% aqueous ethanol and has a pH of between about 5.5 and about 5.7.

In one embodiment of the invention, the liquid pharmaceutical composition comprises at least 15, at least 20 or at least 25 mg/ml of sildenafil citrate and sucralose and between about 40% and about 60% aqueous ethanol and has a pH of about 5.5. In another embodiment of the invention, the liquid pharmaceutical composition comprises about 15, about 20 or about 25 mg/ml of sildenafil citrate and sucralose and between about 40% and about 60% aqueous ethanol and has a pH of about 5.5.

In one embodiment of the invention, the liquid pharmaceutical composition comprises at least 15, at least 20 or at least 25 mg/ml of sildenafil citrate, between about 1 and about 20 mg/ml of sucralose and between about 40% and about 60% aqueous ethanol and has a pH of about 5.5. In another embodiment of the invention, the liquid pharmaceutical composition comprises about 15, about 20 or about 25 mg/ml of sildenafil citrate, between about 1 and about 20 mg/ml of sucralose and between about 40% and about 60% aqueous ethanol and has a pH of about 5.5.

In one embodiment of the invention, the liquid pharmaceutical composition comprises at least 15, at least 20 or at least 25 mg/ml of sildenafil citrate and sucralose and between about 40% and about 60% aqueous ethanol and has a pH of about 5.6. In another embodiment of the invention, the liquid pharmaceutical composition comprises about 15, about 20 or about 25 mg/ml of sildenafil citrate and sucralose and between about 40% and about 60% aqueous ethanol and has a pH of about 5.6.

In one embodiment of the invention, the liquid pharmaceutical composition comprises at least 15, at least 20 or at least 25 mg/ml of sildenafil citrate, between about 1 and about 20 mg/ml of sucralose and between about 40% and about 60% aqueous ethanol and has a pH of about 5.6. In another embodiment of the invention, the liquid pharmaceutical composition comprises about 15, about 20 or about 25 mg/ml of sildenafil citrate, between about 1 and about 20 mg/ml of sucralose and between about 40% and about 60% aqueous ethanol and has a pH of about 5.6.

In one embodiment of the invention, the liquid pharmaceutical composition comprises at least 15, at least 20 or at least 25 mg/ml of sildenafil citrate and sucralose and between about 40% and about 60% aqueous ethanol and has a pH of about 5.7. In another embodiment of the invention, the liquid pharmaceutical composition comprises about 15, about 20 or about 25 mg/ml of sildenafil citrate and sucralose and between about 40% and about 60% aqueous ethanol and has a pH of about 5.7.

In one embodiment of the invention, the liquid pharmaceutical composition comprises at least 15, at least 20 or at least 25 mg/ml of sildenafil citrate, between about 1 and about 20 mg/ml of sucralose and between about 40% and about 60% aqueous ethanol and has a pH of about 5.7. In another embodiment of the invention, the liquid pharmaceutical composition comprises about 15, about 20 or about 25 mg/ml of sildenafil citrate, between about 1 and about 20 mg/ml of sucralose and between about 40% and about 60% aqueous ethanol and has a pH of about 5.7.

In one embodiment of the invention, the liquid pharmaceutical composition comprises at least 10, at least 15, at least 20 or at least 25 mg/ml of sildenafil citrate and sucralose and about 50% aqueous ethanol and has a pH of between about 5.5 and about 5.7. In another embodiment of the invention, the liquid pharmaceutical composition comprises about 15, about 20 or about 25 mg/ml of sildenafil citrate and sucralose and about 50% aqueous ethanol and has a pH of between about 5.5 and about 5.7.

In one embodiment of the invention, the liquid pharmaceutical composition comprises at least 10, at least 15, at least 20 or at least 25 mg/ml of sildenafil citrate, between about 1 and about 20 mg/ml of sucralose and about 50% aqueous ethanol and has a pH of between about 5.5 and about 5.7. In another embodiment of the invention, the liquid pharmaceutical composition comprises about 15, about 20 or about 25 mg/ml of sildenafil citrate, between about 1 and about 20 mg/ml of sucralose and about 50% aqueous ethanol and has a pH of between about 5.5 and about 5.7.

In one embodiment of the invention, the liquid pharmaceutical composition comprises at least 15, at least 20 or at least 25 mg/ml of sildenafil citrate and sucralose and about 50% aqueous ethanol and has a pH of about 5.5. In another embodiment of the invention, the liquid pharmaceutical composition comprises about 15, about 20 or about 25 mg/ml of sildenafil citrate and sucralose and about 50% aqueous ethanol and has a pH of about 5.5.

In one embodiment of the invention, the liquid pharmaceutical composition comprises at least 15, at least 20 or at least 25 mg/ml of sildenafil citrate, between about 1 and about 20 mg/ml of sucralose and about 50% aqueous ethanol and has a pH of about 5.5. In another embodiment of the invention, the liquid pharmaceutical composition comprises about 15, about 20 or about 25 mg/ml of sildenafil citrate, between about 1 and about 20 mg/ml of sucralose and about 50% aqueous ethanol and has a pH of about 5.5.

In one embodiment of the invention, the liquid pharmaceutical composition comprises at least 15, at least 20 or at least 25 mg/ml of sildenafil citrate and sucralose and about 50% aqueous ethanol and has a pH of about 5.6. In another embodiment of the invention, the liquid pharmaceutical composition comprises about 15, about 20 or about 25 mg/ml of sildenafil citrate and sucralose and about 50% aqueous ethanol and has a pH of about 5.6.

In one embodiment of the invention, the liquid pharmaceutical composition comprises at least 15, at least 20 or at least 25 mg/ml of sildenafil citrate, between about 1 and about 20 mg/ml of sucralose and about 50% aqueous ethanol and has a pH of about 5.6. In another embodiment of the invention, the liquid pharmaceutical composition comprises about 15, about 20 or about 25 mg/ml of sildenafil citrate, between about 1 and about 20 mg/ml of sucralose and about 50% aqueous ethanol and has a pH of about 5.6.

In one embodiment of the invention, the liquid pharmaceutical composition comprises at least 15, at least 20 or at least 25 mg/ml of sildenafil citrate and sucralose and about 50% aqueous ethanol and has a pH of about 5.7. In another embodiment of the invention, the liquid pharmaceutical composition comprises about 15, about 20 or about 25 mg/ml of sildenafil citrate and sucralose and about 50% aqueous ethanol and has a pH of about 5.7.

In one embodiment of the invention, the liquid pharmaceutical composition comprises at least 15, at least 20 or at least 25 mg/ml of sildenafil citrate, between about 1 and about 20 mg/ml of sucralose and about 50% aqueous ethanol and has a pH of about 5.7. In another embodiment of the invention, the liquid pharmaceutical composition comprises about 15, about 20 or about 25 mg/ml of sildenafil citrate, between about 1 and about 20 mg/ml of sucralose and about 50% aqueous ethanol and has a pH of about 5.7.

In one embodiment of the invention, the liquid pharmaceutical compositions comprise about 1 to about 20 mg/ml of sucralose and about 1 to about 20 mg/ml of a glycyrrhizinate. As used herein, the term ‘glycyrrhizinate’ refers to a pharmaceutically acceptable salt of glycyrrhizinic acid. Examples of pharmaceutically acceptable salts of glycyrrhizinic acid include disodium glycyrrhizinate, dipotassium glycyrrhizinate (DPG), monoammonium glycyrrhizinate (MAG), triammonium glycyrrhizinate, monopotassium glycyrrhizinate, tripotassium glycyrrhizinate, monosodium glycyrrhizinate and trisodium glycyrrhizinate. In a preferred embodiment, the pharmaceutically acceptable salt of glycyrrhizinic acid comprises monoammonium glycyrrhizinate. In another preferred embodiment, the pharmaceutically acceptable salt of glycyrrhizinic acid comprises dipotassium glycyrrhizinate. In one embodiment of the invention, the liquid pharmaceutical compositions comprise about 1 to about 20 mg/ml of sucralose and about 1, about 2, about 3, about 4, about 5, about 6, about 7, about 8, about 9, about 10, about 11, about 12, about 13, about 14, about 15, about 16, about 17, about 18, about 19 about 1, or about 20 mg/ml of a glycyrrhizinate. In another embodiment of the invention, the liquid pharmaceutical compositions comprise about 1 to about 20 mg/ml of sucralose and do not comprise a glycyrrhizinate.

In one embodiment of the invention, the liquid pharmaceutical composition comprises at least 15 mg/ml of sildenafil citrate, sucralose and monoammonium or dipotassium glycyrrhizinate, and has a pH of between about 5.5 and about 5.7.

In one embodiment of the invention, the liquid pharmaceutical composition comprises at least 15, at least 20 or at least 25 mg/ml of sildenafil citrate, sucralose, about 1 to about 20 mg/ml of a glycyrrhizinate and between about 40% and about 60% aqueous ethanol and has a pH of between about 5.5 and about 5.7. In another embodiment of the invention, the liquid pharmaceutical composition comprises about 15, about 20 or about 25 mg/ml of sildenafil citrate, sucralose, about 1 to about 20 mg/ml of a glycyrrhizinate and between about 40% and about 60% aqueous ethanol and has a pH of between about 5.5 and about 5.7.

In one embodiment of the invention, the liquid pharmaceutical composition comprises at least 15, at least 20 or at least 25 mg/ml of sildenafil citrate, between about 1 and about 20 mg/ml of sucralose, about 1 to about 20 mg/ml of a glycyrrhizinate and between about 40% and about 60% aqueous ethanol and has a pH of between about 5.5 and about 5.7. In another embodiment of the invention, the liquid pharmaceutical composition comprises about 15, about 20 or about 25 mg/ml of sildenafil citrate, between about 1 and about 20 mg/ml of sucralose, about 1 to about 20 mg/ml of a glycyrrhizinate and between about 40% and about 60% aqueous ethanol and has a pH of between about 5.5 and about 5.7.

In one embodiment of the invention, the liquid pharmaceutical composition comprises at least 15, at least 20 or at least 25 mg/ml of sildenafil citrate, sucralose, about 1 to about 20 mg/ml of a glycyrrhizinate and between about 40% and about 60% aqueous ethanol and has a pH of about 5.5. In another embodiment of the invention, the liquid pharmaceutical composition comprises about 15, about 20 or about 25 mg/ml of sildenafil citrate, sucralose, about 1 to about 20 mg/ml of a glycyrrhizinate and between about 40% and about 60% aqueous ethanol and has a pH of about 5.5.

In one embodiment of the invention, the liquid pharmaceutical composition comprises at least 15, at least 20 or at least 25 mg/ml of sildenafil citrate, between about 1 and about 20 mg/ml of sucralose, about 1 to about 20 mg/ml of a glycyrrhizinate and between about 40% and about 60% aqueous ethanol and has a pH of about 5.5. In another embodiment of the invention, the liquid pharmaceutical composition comprises about 15, about 20 or about 25 mg/ml of sildenafil citrate, between about 1 and about 20 mg/ml of sucralose, about 1 to about 20 mg/ml of a glycyrrhizinate and between about 40% and about 60% aqueous ethanol and has a pH of about 5.5.

In one embodiment of the invention, the liquid pharmaceutical composition comprises at least 15, at least 20 or at least 25 mg/ml of sildenafil citrate, sucralose, about 1 to about 20 mg/ml of a glycyrrhizinate and between about 40% and about 60% aqueous ethanol and has a pH of about 5.6. In another embodiment of the invention, the liquid pharmaceutical composition comprises about 15, about 20 or about 25 mg/ml of sildenafil citrate, sucralose, about 1 to about 20 mg/ml of a glycyrrhizinate and between about 40% and about 60% aqueous ethanol and has a pH of about 5.6.

In one embodiment of the invention, the liquid pharmaceutical composition comprises at least 15, at least 20 or at least 25 mg/ml of sildenafil citrate, between about 1 and about 20 mg/ml of sucralose, about 1 to about 20 mg/ml of a glycyrrhizinate and between about 40% and about 60% aqueous ethanol and has a pH of about 5.6. In another embodiment of the invention, the liquid pharmaceutical composition comprises about 15, about 20 or about 25 mg/ml of sildenafil citrate, between about 1 and about 20 mg/ml of sucralose, about 1 to about 20 mg/ml of a glycyrrhizinate and between about 40% and about 60% aqueous ethanol and has a pH of about 5.6.

In one embodiment of the invention, the liquid pharmaceutical composition comprises at least 15, at least 20 or at least 25 mg/ml of sildenafil citrate, sucralose, about 1 to about 20 mg/ml of a glycyrrhizinate and between about 40% and about 60% aqueous ethanol and has a pH of about 5.7. In another embodiment of the invention, the liquid pharmaceutical composition comprises about 15, about 20 or about 25 mg/ml of sildenafil citrate, sucralose, about 1 to about 20 mg/ml of a glycyrrhizinate and between about 40% and about 60% aqueous ethanol and has a pH of about 5.7.

In one embodiment of the invention, the liquid pharmaceutical composition comprises at least 15, at least 20 or at least 25 mg/ml of sildenafil citrate, between about 1 and about 20 mg/ml of sucralose, about 1 to about 20 mg/ml of a glycyrrhizinate and between about 40% and about 60% aqueous ethanol and has a pH of about 5.7. In another embodiment of the invention, the liquid pharmaceutical composition comprises about 15, about 20 or about 25 mg/ml of sildenafil citrate, between about 1 and about 20 mg/ml of sucralose, about 1 to about 20 mg/ml of a glycyrrhizinate and between about 40% and about 60% aqueous ethanol and has a pH of about 5.7.

In one embodiment of the invention, the liquid pharmaceutical composition comprises at least 15, at least 20 or at least 25 mg/ml of sildenafil citrate, sucralose, about 1 to about 20 mg/ml of a glycyrrhizinate and about 50% aqueous ethanol and has a pH of between about 5.5 and about 5.7. In another embodiment of the invention, the liquid pharmaceutical composition comprises about 15, about 20 or about 25 mg/ml of sildenafil citrate, sucralose, about 1 to about 20 mg/ml of a glycyrrhizinate and about 50% aqueous ethanol and has a pH of between about 5.5 and about 5.7.

In one embodiment of the invention, the liquid pharmaceutical composition comprises at least 15, at least 20 or at least 25 mg/ml of sildenafil citrate, between about 1 and about 20 mg/ml of sucralose, about 1 to about 20 mg/ml of a glycyrrhizinate and about 50% aqueous ethanol and has a pH of between about 5.5 and about 5.7. In another embodiment of the invention, the liquid pharmaceutical composition comprises about 15, about 20 or about 25 mg/ml of sildenafil citrate, between about 1 and about 20 mg/ml of sucralose, about 1 to about 20 mg/ml of a glycyrrhizinate and about 50% aqueous ethanol and has a pH of between about 5.5 and about 5.7.

In one embodiment of the invention, the liquid pharmaceutical composition comprises at least 15, at least 20 or at least 25 mg/ml of sildenafil citrate, sucralose, about 1 to about 20 mg/ml of a glycyrrhizinate and about 50% aqueous ethanol and has a pH of about 5.5. In another embodiment of the invention, the liquid pharmaceutical composition comprises about 15, about 20 or about 25 mg/ml of sildenafil citrate, sucralose, about 1 to about 20 mg/ml of a glycyrrhizinate and about 50% aqueous ethanol and has a pH of about 5.5.

In one embodiment of the invention, the liquid pharmaceutical composition comprises at least 15, at least 20 or at least 25 mg/ml of sildenafil citrate, between about 1 and about 20 mg/ml of sucralose, about 1 to about 20 mg/ml of a glycyrrhizinate and about 50% aqueous ethanol and has a pH of about 5.5. In another embodiment of the invention, the liquid pharmaceutical composition comprises about 15, about 20 or about 25 mg/ml of sildenafil citrate, between about 1 and about 20 mg/ml of sucralose, about 1 to about 20 mg/ml of a glycyrrhizinate and about 50% aqueous ethanol and has a pH of about 5.5.

In one embodiment of the invention, the liquid pharmaceutical composition comprises at least 15, at least 20 or at least 25 mg/ml of sildenafil citrate, sucralose, about 1 to about 20 mg/ml of a glycyrrhizinate and about 50% aqueous ethanol and has a pH of about 5.6. In another embodiment of the invention, the liquid pharmaceutical composition comprises about 15, about 20 or about 25 mg/ml of sildenafil citrate, sucralose, about 1 to about 20 mg/ml of a glycyrrhizinate and about 50% aqueous ethanol and has a pH of about 5.6.

In one embodiment of the invention, the liquid pharmaceutical composition comprises at least 15, at least 20 or at least 25 mg/ml of sildenafil citrate, between about 1 and about 20 mg/ml of sucralose, about 1 to about 20 mg/ml of a glycyrrhizinate and about 50% aqueous ethanol and has a pH of about 5.6. In another embodiment of the invention, the liquid pharmaceutical composition comprises about 15, about 20 or about 25 mg/ml of sildenafil citrate, between about 1 and about 20 mg/ml of sucralose, about 1 to about 20 mg/ml of a glycyrrhizinate and about 50% aqueous ethanol and has a pH of about 5.6.

In one embodiment of the invention, the liquid pharmaceutical composition comprises at least 15, at least 20 or at least 25 mg/ml of sildenafil citrate, sucralose, about 1 to about 20 mg/ml of a glycyrrhizinate and about 50% aqueous ethanol and has a pH of about 5.7. In another embodiment of the invention, the liquid pharmaceutical composition comprises about 15, about 20 or about 25 mg/ml of sildenafil citrate, sucralose, about 1 to about 20 mg/ml of a glycyrrhizinate and about 50% aqueous ethanol and has a pH of about 5.7.

In one embodiment of the invention, the liquid pharmaceutical composition comprises at least 15, at least 20 or at least 25 mg/ml of sildenafil citrate, between about 1 and about 20 mg/ml of sucralose, about 1 to about 20 mg/ml of a glycyrrhizinate and about 50% aqueous ethanol and has a pH of about 5.7. In another embodiment of the invention, the liquid pharmaceutical composition comprises about 15, about 20 or about 25 mg/ml of sildenafil citrate, between about 1 and about 20 mg/ml of sucralose, about 1 to about 20 mg/ml of a glycyrrhizinate and about 50% aqueous ethanol and has a pH of about 5.7.

In one embodiment of the invention, the liquid pharmaceutical compositions also comprise a flavoring agent other than sucralose. Examples of flavoring agents include, but are not limited to, essential oils (e.g. lemon oil), sweeteners (e.g. sugars or sugar substitutes), vanilla extract, peppermint extract, wintergreen extract, eucalyptus extract, mint extract, cinnamon, chocolate extract and rum extract, glutamates, esters and aldehydes.

In one embodiment of the invention, the liquid pharmaceutical compositions also comprise a bitterness blocking agent. Examples of bitterness blocking include, but are not limited to, essential oils of tarragon (e.g., Artemisia dracunculus) and/or basil (e.g., Ocium basilicum).

The present invention also relates to liquid pharmaceutical compositions comprising at least 15 mg/ml of sildenafil citrate, having a pH of between about 5.5 and about 5.7 and which have a shelf-life stability of at least 12 months. As used herein, the terms ‘stable’, ‘stability’ and ‘shelf-life’ shall be understood according to the definitions accepted by the US Food and Drug Agency (FDA) as provided within the International Council on Harmonization's guidance documents (ICH) Q1(A-F) and QSC.

In one embodiment of the invention, the liquid pharmaceutical compositions have a shelf-life stability of at least 12 months. In another the liquid pharmaceutical compositions have a shelf-life stability of at least 18 months. In another the liquid pharmaceutical compositions have a shelf-life stability of at least 24 months. In another the liquid pharmaceutical compositions have a shelf-life stability of at least 30 months. In another the liquid pharmaceutical compositions have a shelf-life stability of at least 36 months.

In one embodiment of the invention, the liquid pharmaceutical compositions comprise less than 1% of impurity B at Day 0, Day 30, Day 180, Day 270, Day 365, Day 730 or Day 1095 after initiation of stability testing. In another embodiment of the invention, the liquid pharmaceutical compositions comprise less than 0.5% of impurity B at Day 0, Day 30, Day 180, Day 270, Day 365, Day 730 or Day 1095 after initiation of stability testing. In another embodiment of the invention, the liquid pharmaceutical compositions comprise less than 0.2% of impurity B at Day 0, Day 30, Day 180, Day 270, Day 365, Day 730 or Day 1095 after initiation of stability testing. In another embodiment of the invention, the liquid pharmaceutical compositions comprise less than 0.15% of impurity B at Day 0, Day 30, Day 180, Day 270, Day 365, Day 730 or Day 1095 after initiation of stability testing. In another embodiment of the invention, the liquid pharmaceutical compositions comprise less than 0.1% of impurity B at Day 0, Day 30, Day 180, Day 270, Day 365, Day 730 or Day 1095 after initiation of stability testing. In another embodiment of the invention, the liquid pharmaceutical compositions comprise less than 0.05% of impurity B at Day 0, Day 30, Day 180, Day 270, Day 365, Day 730 or Day 1095 after initiation of stability testing.

The present invention also relates to oral dosage forms comprising a liquid pharmaceutical composition of at least 15 mg/ml of sildenafil citrate and having a pH of between about 5.5 and about 5.7.

In one embodiment of the invention, the oral dosage form is a liquid. Examples of a liquid oral dosage form include, but are not limited to, sprays, solutions and drops. In another embodiment of the invention, the oral dosage form incorporates the liquid pharmaceutical compositions of the invention into a non-liquid dosage form, for example by including a liquid core, comprising the pharmaceutical composition of the invention, within an outer coating or by impregnating a solid material with the pharmaceutical composition of the invention. Examples of a non-liquid dosage forms include, but are not limited to, gelcaps, capsules and chewing gums.

The present invention also relates to buccal, sublingual or inhaled dosage forms comprising a liquid pharmaceutical composition of at least 15 mg/ml of sildenafil citrate and having a pH of between about 5.5 and about 5.7.

In one embodiment of the invention, the buccal, sublingual or inhaled dosage form is a liquid. Examples of a liquid oral dosage form include, but are not limited to, solutions and sprays. In another embodiment of the invention, the buccal or sublingual dosage form incorporates the liquid pharmaceutical compositions of the invention into a non-liquid dosage form, for example by impregnating a solid material with the pharmaceutical composition of the invention. Examples of a non-liquid dosage forms include, but are not limited to, thin-films.

Chewing gums, sprays and thin-films containing sildenafil citrate are described at least in U.S. Pat. Nos. 6,531,114, 6,592,850, 7,556,487, 9,370,518 and 10,092,651, U.S. Patent application publications 20100203191, 20100209553, 20130143894 and international patent application publication WO2013085224, each of which is fully incorporated by reference.

The present invention also relates to methods of treating erectile dysfunction comprising administering, to a human subject in need thereof, a liquid pharmaceutical composition of at least 15 mg/ml of sildenafil citrate and having a pH of between about 5.5 and about 5.7.

In one embodiment of the invention, the method of treating erectile dysfunction comprises administering, to a human subject in need thereof, a liquid pharmaceutical composition of at least 15 mg/ml of sildenafil citrate and having a pH of between about 5.5 and about 5.7. In a preferred embodiment of the invention, the volume of the liquid pharmaceutical composition administered to said subject is less than 10 ml. In a more preferred embodiment of the invention, the volume of the liquid pharmaceutical composition administered to said subject is less than 7 ml. In a most preferred embodiment of the invention, the volume of the liquid pharmaceutical composition administered to said subject is less than 5 ml.

The use of such high concentration, low volume, liquid pharmaceutical compositions of sildenafil citrate to treat erectile dysfunction allows for the rapid absorption of sildenafil with an improved relative bioavailability when compared to conventional solid dosage forms of the drug. As demonstrated in U.S. Pat. No. 9,968,609, such an improvement can allow for a more rapid onset of therapeutic effect than conventional solid dosage forms, even at lower comparative doses.

The present invention also relates to methods for the manufacture of liquid pharmaceutical compositions comprising at least 15 mg/ml of sildenafil citrate and between about 40% and about 60% aqueous ethanol and having a pH of between about 5.5 and about 5.7, comprising adjusting the pH of the aqueous ethanol to between about 5.5 and about 5.7 and then mixing, at room temperature, said aqueous ethanol together with sildenafil citrate.

In one embodiment of the invention, the method for the manufacture of liquid pharmaceutical compositions comprising at least 15 mg/ml of sildenafil citrate and between about 40% and about 60% aqueous ethanol and having a pH of between about 5.5 and about 5.7, comprises adjusting the pH of the aqueous ethanol to between about 5.5 and about 5.7 and then mixing, at room temperature, said aqueous ethanol together with sildenafil citrate.

In one embodiment of the invention, the method for the manufacture of liquid pharmaceutical compositions comprising at least 15 mg/ml of sildenafil citrate, sucralose and between about 40% and about 60% aqueous ethanol and having a pH of between about 5.5 and about 5.7, comprises adjusting the pH of the aqueous ethanol to between about 5.5 and about 5.7 and then mixing, at room temperature, said aqueous ethanol together with sucralose and sildenafil citrate.

It will readily be appreciated by one of skill in the art, that, by being available at room temperature, the methods of manufacture of the present invention represent a distinct and surprising advantage over the known methods of preparing aqueous ethanol liquid compositions of sildenafil citrate, such as those demonstrated in U.S. Patent 9,968,609, which rely on heating the ethanol to potentially volatile, high temperatures before introducing the sildenafil citrate and which risk precipitation of the sildenafil when their compositions cool to room temperature.

This invention will be better understood by reference to the Examples, which follow. Those of skill in the art will readily appreciate that the specific experiments detailed are only illustrative of the invention as described more fully in the claims which follow thereafter.

### EXAMPLES

### Example 1

### Dissolution and Stability of Sildenafil Base at Different pHs

### Example 1a

840 mg of sildenafil base (Carbosynth) was introduced into 40 ml of 50% aqueous ethanol at an unadjusted pH of 7.7. After stirring for 10 minutes, the sildenafil remained undissolved. The pH of the mixture was then lowered to 5.6 by the introduction of 1M HCl and stirred again for another 10 minutes with the mixture remaining opaque and undissolved. Finally, the pH of the mixture was further lowered to a pH of approximately 4 by the introduction of additional 1M HCl upon which the mixture began to solubilize. The mixture was stirred at pH 4.25 for 1 hour upon which the solution was found to be totally transparent and without precipitate, at a pH of 4.28.

### Example 1b

840 mg of sildenafil base (Carbosynth), together with 840 mg of sucralose was introduced into 40 ml of 50% aqueous ethanol at an unadjusted pH of 7.7. After stirring for 10 minutes, the sildenafil remained undissolved. The pH of the mixture was then lowered to 5.6 by the introduction of 1M HCl and stirred again for another 10 minutes with the mixture remaining opaque and undissolved. The pH of the mixture was further lowered to a pH of approximately 4.6 by the introduction of additional 1M HCl upon which the mixture began to solubilize. Additional 1M HCl was added to the mixture until it completely solubilized at a pH of 3.83.

### Example 1c

945 mg of sildenafil base (Carbosynth), together with 945 mg of sucralose was introduced into 45 ml of 50% aqueous ethanol at an unadjusted pH of 7.7. After stirring for 10 minutes, the sildenafil remained undissolved. The pH of the mixture was then lowered to by the stepwise introduction of 1M HCl during stirring until the mixture was fully solubilized at pH 4.92.

### Example 2

### Dissolution and Stability of Sildenafil Citrate at Different pHs

At room temperature, a phosphate buffer was prepared by weighing 3.9 g of NaH₂PO_{4.2}H₂O into a 500 mL volumetric flask and adding approximately 300 mL of purified water until the phosphate fully dissolved. The pH was then adjusted to 7 with the introduction of NaOH and dilute to the requisite volume by the further addition of purified water.

A 50% ethanol mixture was prepared by introducing either purified water, taken from a MilliQ™ unit or phosphate buffer, to equivalent volumes of Ph.Eur. grade absolute alcohol. The pH of the resultant mixture was then adjusted by using either NaOH 1N solutions and/or Sodium Citrate 1N solutions to reach predetermined pH values.

Samples of sucralose were then dissolved in the pH adjusted 50% ethanol, whilst stirred with a magnetic stirrer and a needle, to a predetermined concentration. After the sucralose was dissolved, samples of sildenafil citrate (Teva Pharmaceuticals) were added to the solution and mixed under stirring, in quantities to match the predetermined concentrations of sucralose. After preparation, the solutions were each filtered through PDVF 0.22 micro filter of 13 mm in size and placed in polypropylene containers of adequate volume.

Before being placed on storage, and at time points 7 and 42 days, the concentration of sildenafil in the mixture was assayed using HPLC. The samples were then stored at either −20° C., 2-8° C. or 25° C. for periods of up to 42 days. Periodically, the samples were visually inspected for evidence of precipitation or crystallization. No breakdown products were identified during the study under any of the conditions tested. The results of the visual inspections are shown in Table 1 and of the assays in Table 2.

TABLE 1

| Concentration | | | | | |
|---|---|---|---|---|---|
| sildenafil | | | | | |
| (mg/ml) | pH | buffer | 25° C. | 2-8° C. | −20° C. |
| 15 | 5.0 | Sodium Citrate | Prec. | Prec. | Prec. |
| 15 | 5.2 | NaOH | — | — | Prec. |
| 18 | | Sodium Citrate | — | — | Prec. |
| 18 | | NaOH | — | — | Prec. |
| 15* | 5.24 | NaOH | — | — | — |
| 21* | 5.29 | NaOH | — | — | Prec. |
| 18* | 5.3 | NaOH | — | — | — |
| 21 | 5.5 | Sodium Citrate | — | — | Prec. |
| 21 | | NaOH | — | — | Prec. |
| 15 | 5.6 | Sodium Citrate | — | — | — |
| 15 | | NaOH | — | — | — |
| 15* | | NaOH | — | — | — |
| 18 | | Sodium Citrate | — | — | — |
| 18 | | NaOH | — | — | — |
| 21 | | Sodium Citrate | — | — | — |
| 21 | | NaOH | — | — | — |
| 21* | | NaOH | — | Prec. | Prec. |
| 18* | 5.63 | NaOH | — | — | — |
| 21 | 5.8 | Sodium Citrate | — | — | — |
| 21 | | NaOH | — | Prec. | — |
| 18 | 6.0 | Sodium Citrate | — | Prec. | Prec. |
| 18* | | NaOH | — | Prec. | Prec. |
| 15* | 6.01 | NaOH | — | Prec. | Prec. |
| 21* | | NaOH | — | Prec. | Prec. |
| 15 | 6.2 | Sodium Citrate | — | Prec. | Prec. |
| 15 | | NaOH | Prec. | Prec. | Prec. |
| 18 | | NaOH | Prec. | Prec. | Prec. |

*50% ethanol/phosphate buffer

TABLE 2

| Concentration | | | | 2-8 ° C. | | 25 ° C. | |
|---|---|---|---|---|---|---|---|
| sildenafil | | | Day | Day | Day | Day | Day |
| (mg/ml) | pH | buffer | 0 | 7 | 42 | 7 | 42 |
| 15 | 5.0 | Sodium | 15.22 | 11.05 | — | 14.89 | — |
| | | Citrate | | | | | |
| 15 | 5.2 | NaOH | 15.05 | 14.99 | — | 15.01 | — |
| 18 | | Sodium | 18.09 | 17.96 | — | 18.02 | — |
| | | Citrate | | | | | |
| 18 | | NaOH | 18.23 | 17.89 | — | 17.93 | — |
| 15* | 5.24 | NaOH | 15.06 | 14.97 | — | 15.01 | — |
| 21* | 5.29 | NaOH | 20.94 | 20.97 | — | 20.98 | — |
| 18* | 5.3 | NaOH | 18.07 | 18.05 | — | 18.07 | — |
| 21 | 5.5 | Sodium | 21.32 | 21.14 | — | 21.22 | — |
| | | Citrate | | | | | |
| 21 | | NaOH | 21.11 | 20.86 | — | 20.97 | — |
| 15 | 5.6 | Sodium | 14.92 | 14.80 | 15.04 | 14.79 | 14.88 |
| | | Citrate | | | | | |
| 15 | | NaOH | 15.14 | 15.03 | 15.05 | 14.93 | 14.97 |
| 15* | | NaOH | 14.96 | 14.92 | — | 14.94 | — |
| 18 | | Sodium | 18.19 | 17.82 | 17.88 | 17.82 | 17.91 |
| | | Citrate | | | | | |
| 18 | | NaOH | 18.13 | 17.86 | 18.07 | 17.86 | 18.07 |
| 21 | | Sodium | 21.26 | 20.99 | 21.41 | 21.06 | 21.27 |
| | | Citrate | | | | | |
| 21 | | NaOH | 21.20 | 21.04 | 21.43 | 21.02 | 21.32 |
| 21* | | NaOH | 20.97 | 20.93 | — | 20.99 | — |
| 18* | 5.63 | NaOH | 18.18 | 18.01 | — | 18.04 | — |
| 21 | 5.8 | Sodium | 21.08 | 20.96 | — | 21.07 | — |
| | | Citrate | | | | | |
| 21 | | NaOH | 21.15 | 20.95 | — | 21.73 | — |
| 18 | 6.0 | Sodium | 17.76 | 14.95 | — | 15.08 | — |
| | | Citrate | | | | | |
| 15* | 6.01 | NaOH | 14.83 | 14.69 | — | 14.83 | — |
| 21* | | NaOH | 20.77 | 16.22 | — | 18.02 | — |
| 15 | 6.2 | Sodium | 14.75 | 11.11 | — | 12.10 | — |
| | | Citrate | | | | | |
| 15 | | NaOH | 15.14 | 10.42 | — | 11.75 | — |

*50% ethanol/phosphate buffer

The samples of 21 mg/ml at a pH of 5.6 were maintained in stability testing at 25° C. for a total of 285 days. The results of the assays for these samples at Day 285 are shown in Table 3.

TABLE 3

| buffer | Sildenafil | Impurity B | Impurity D |
|---|---|---|---|
| Sodium Citrate | 21.14 | 0.01 | 0.02 |
| NaOH | 21.16 | 0.00 | 0.02 |

### Example 3

### Ionic Strength Studies

To attempt to analyse whether the stabilization effects seen in the solubility studies were as a result of ionic strength rather than pH, samples of sildenafil citrate and sucralose (in equal concentrations) were dissolved in of Ph.Eur. grade absolute alcohol either directly mixed with equivalent volumes of either NaCl aqueous solutions or purified water or by being introduced to 40 ml of the diluent, stirred and then having an additional 10 ml of diluent added. The mixtures were then either placed on storage or NaOH 1N solution was introduced to reach a predetermined pH values. Before being placed on storage, and at time points 7 and 42 days, the concentration of sildenafil in the mixtures were assayed using HPLC. The samples were then stored at either −20° C., 2-8° C. or 25° C. for periods of up to 42 days. Periodically, the samples were visually inspected for evidence of crystallization. The results of the visual inspections are shown in Table 4 and of the assays in Table 5.

TABLE 4

| Concentration | | | | | |
|---|---|---|---|---|---|
| sildenafil | | NaCl diluent | | | |
| (mg/ml) | pH | (mM) | 25° C. | 2-8° C. | −20° C. |
| 15 | 4.43 | 100 | — | — | — |
| 15* | 4.39 | 100 | — | — | Prec. |
| 15 | 4.45 | 50 | — | — | Prec. |
| 15 | 4.52 | 25 | — | Prec. | Prec. |
| 15 | 4.75 | purified water | — | Prec. | Prec. |
| 15** | 5.5 | 100 | — | — | — |
| 18** | 5.5 | 100 | — | — | Prec. |
| 21** | 5.5 | 100 | — | — | Prec. |

*sildenafil + sucralose initially added to 40 ml of diluent
**NaOH

TABLE 5

| Concentration | | | | 2-8 ° C. | | 25 ° C. | |
|---|---|---|---|---|---|---|---|
| sildenafil | | NaCl diluent | Day | Day | Day | Day | Day |
| (mg/ml) | pH | (mM) | 0 | 7 | 42 | 7 | 42 |
| 15 | 4.43 | 100 | 15.59 | 15.65 | 15.84 | 15.76 | 15.81 |
| 15* | 4.39 | 100 | 15.13 | 15.31 | 14.66 | 15.38 | 15.45 |
| 15 | 4.45 | 50 | 15.35 | 15.26 | — | 15.60 | — |
| 15 | 4.52 | 25 | 15.32 | 9.88 | — | 15.50 | — |
| 15 | 4.75 | purified | 13.38 | 8.21 | — | 13.59 | — |
| | | water | | | | | |
| 15** | 5.5 | 100 | 15.13 | 15.18 | — | 15.15 | — |
| 18** | 5.5 | 100 | 18.13 | 17.48 | 16.41 | 17.38 | 17.54 |
| 21** | 5.5 | 100 | 21.07 | — | 19.75 | 20.07 | 20.20 |

*sildenafil + sucralose initially added to 40 ml of diluent
**NaOH

### Example 4

### Dissolution and Stability of Sildenafil Citrate at Different Concentrations of Ethanol

Samples of sildenafil citrate, with, or without equivalent concentrations of sucralose, were added to buffered 60% aqueous ethanol. Before being placed on storage, and at time points 7 and 42 days, the concentration of sildenafil in the mixtures were assayed using HPLC. The samples were then stored at either −20° C., 2-8° C. or 25° C. for periods of up to 42 days. Periodically, the samples were visually inspected for evidence of crystallization. The results of the visual inspections are shown in Table 6 and of the assays in Table 7.

TABLE 6

| Concentration | | | | | |
|---|---|---|---|---|---|
| sildenafil | | | | | |
| (mg/ml) | pH | buffer | 25° C. | 2-8° C. | −20° C. |
| 18 | 5.5 | NaOH | — | Prec. | Prec. |
| 18* | 5.5 | NaOH | — | Prec. | Prec. |
| 21 | 5.5 | NaOH | — | Prec. | Prec. |

*with sucralose

TABLE 7

| Concentration | | | | 2-8 ° C. | | 25 ° C. | |
|---|---|---|---|---|---|---|---|
| sildenafil | | | Day | Day | Day | Day | Day |
| (mg/ml) | pH | buffer | 0 | 7 | 42 | 7 | 42 |
| 18 | 5.5 | NaOH | 17.42 | 18.33 | 15.23 | 18.08 | 17.19 |
| 18* | 5.5 | NaOH | 17.56 | 18.55 | 15.24 | 18.00 | 17.94 |
| 21 | 5.5 | NaOH | 20.52 | 22.11 | 13.78 | 20.40 | 19.61 |

*with sucralose

### Example 5

### Maximum Dissolution and Stability of Sildenafil Citrate Study

To attempt to identify the maximum dissolution of sildenafil citrate, it, with or without equivalent concentrations of sucralose, was added to 50% aqueous ethanol until precipitation occurred. Before being placed on storage, and at time points 7, 42 days and 2 months, the concentration of sildenafil in the mixtures were assayed using HPLC. The samples were then stored at either −20° C., 2-8° C. or 25° C. for periods of up to 2 months. Periodically, the samples were visually inspected for evidence of crystallization. The results of the visual inspections are shown in Table 8 and of the assays in Table 9.

TABLE 8

| Day 0 | | | | | |
|---|---|---|---|---|---|
| concentration | | | | | |
| sildenafil | | | | | |
| (mg/ml) | pH | buffer | 25° C. | 2-8° C. | −20° C. |
| 51.65 | 5.5 | NaOH | — | Prec. | Prec. |
| 82.79* | 5.5 | NaOH | — | Prec. | Prec. |

| | | | |
|---|---|---|---|
| 123.95* | 5.5 | NaOH | Prec. |

*with sucralose

TABLE 9

| Day 0 | | | | | | | |
|---|---|---|---|---|---|---|---|
| concentration | | | 2-8 ° C. | | 25 ° C. | | |
| sildenafil | | | Day | Day | Day | Day | 2 |
| (mg/ml) | pH | buffer | 7 | 42 | 7 | 42 | months |
| 51.65 | 5.5 | NaOH | 54.00 | 33.50 | 53.22 | 49.62 | — |
| 82.79* | 5.5 | NaOH | 84.56 | 27.12 | 84.58 | 87.82 | 68.56 |
| 123.95* | 5.5 | NaOH | — | — | 61.02 | — | — |

*with sucralose

### Example 6

### Extended Stability of Sildenafil Citrate in Ethanol

Samples of sildenafil citrate, sucralose, monoammonium glycyrrhizinate and eucalyptus-menthol (Symrise #745670) were added to buffered 50% ethanol to reach concentrations of 20 mg/ml, 1 mg/ml, 1 mg/ml and 3 mg/ml respectively. Before being placed on storage in glass vials, plastic containers or Easysnap® blisters, and at time points up to 160 days, the concentration of sildenafil and its impurities in the mixtures were assayed using HPLC. The samples were then stored at 25° C./60% RH, 30° C./65% RH or 40° C./75% RH for periods of up to 160 days. The results of the assays are shown in Table 10 (sildenafil), Table 11 (Impurity B) and Table 12 (Impurity D).

TABLE 10

| | | % LS | | | |
|---|---|---|---|---|---|
| container | conditions | Day 0 | Day 32 | Day 80 | Day 159 |
| glass | 25° C./60% RH | 102.02 | 100.76 | 101.47 | 100.68 |
| | 30° C./65% RH | | 100.46 | — | — |
| | 40° C./75% RH | | 100.39 | — | 100.60 |
| plastic | 25° C./60% RH | | 100.42 | 101.59 | 101.19 |
| | 30° C./65% RH | | 100.79 | — | — |
| | 40° C./75% RH | | 100.76 | — | — |
| Easysnap ® | 25° C./60% RH | | 100.87 | 101.53 | 100.91 |
| | 30° C./65% RH | | 100.85 | — | — |
| | 40° C./75% RH | | 101.46 | — | — |

TABLE 11

| | | % RS | | | |
|---|---|---|---|---|---|
| container | conditions | Day 0 | Day 32 | Day 80 | Day 159 |
| glass | 25° C./60% RH | | 0.07 | 0.10 | 0.17 |
| | 30° C./65% RH | | 0.10 | — | — |
| | 40° C./75% RH | | 0.17 | — | 0.45 |
| plastic | 25° C./60% RH | | 0.08 | 0.09 | 0.16 |
| | 30° C./65% RH | | 0.09 | — | — |
| | 40° C./75% RH | | 0.17 | — | — |
| Easysnap ® | 25° C./60% RH | | 0.09 | 0.11 | 0.20 |
| | 30° C./65% RH | | 0.10 | — | — |
| | 40° C./75% RH | | 0.20 | — | — |

TABLE 12

| | | % RS | | | |
|---|---|---|---|---|---|
| container | conditions | Day 0 | Day 32 | Day 80 | Day 159 |
| glass | 25° C./60% RH | | 0.030 | 0.030 | 0.038 |
| | 30° C./65% RH | | 0.030 | — | — |
| | 40° C./75% RH | | 0.030 | — | 0.039 |
| plastic | 25° C./60% RH | | 0.040 | 0.030 | 0.033 |
| | 30° C./65% RH | | 0.030 | — | — |
| | 40° C./75% RH | | 0.030 | — | — |
| Easysnap ® | 25° C./60% RH | | 0.030 | 0.030 | 0.034 |
| | 30° C./65% RH | | 0.030 | — | — |
| | 40° C./75% RH | | 0.030 | — | — |

### Example 7

### Stability of Sildenafil Citrate in Ethanol and Additional Excipients

Samples of sildenafil citrate and sucralose were added to buffered 50% ethanol and eucalyptus and/or a glycyrrhizinate (monoammonium or dipotassium) to reach concentrations of 20 mg/ml sildenafil citrate, 1 mg/ml sucralose and 3 mg/ml eucalyptus and/or 1 mg/ml of a glycyrrhizinate (monoammonium or dipotassium) and stored for at least 30 days at either 25° C. or 30° C. Before being placed on storage and at 37 days, the mixture's pH was measured and the concentration of sildenafil and its impurities in the mixtures were assayed using HPLC. The results of the assays at 37 days are shown in Table 13 (sildenafil) and Table 14 (impurities).

TABLE 13

| | | Temperature/ | Mean |
|---|---|---|---|
| Test composition | pH | ° C. | % LS |
| Sildenafil citrate + sucralose | 5.55 | 25 | 100.24 |
| | — | 30 | 99.69 |
| Sildenafil citrate, sucralose + | 5.58 | 25 | 99.39 |
| monoammonium glycyrrhizinate | — | 30 | 99.80 |
| Sildenafil citrate, sucralose + | 5.56 | 25 | 100.04 |
| dipotassium glycyrrhizinate | — | 30 | 99.65 |
| Sildenafil citrate, sucralose, | 5.59 | 25 | 98.84 |
| monoammonium glycyrrhizinate + | — | 30 | 99.14 |
| eucalyptus | | | |
| Sildenafil citrate, sucralose, dipotassium | 5.59 | 25 | 98.96 |
| glycyrrhizinate + eucalyptus | — | 30 | 99.54 |

TABLE 14

| | Temperature/ | Mean % RS | |
|---|---|---|---|
| Test composition | ° C. | Imp B | Imp D |
| Sildenafil citrate + sucralose | 25 | ND | 0.024 |
| | 30 | 0.017 | 0.027 |
| Sildenafil citrate, sucralose + | 25 | ND | 0.025 |
| monoammonium glycyrrhizinate | 30 | ND | 0.027 |
| Sildenafil citrate, sucralose + | 25 | ND | 0.026 |
| dipotassium glycyrrhizinate | 30 | ND | 0.027 |
| Sildenafil citrate, sucralose, | 25 | 0.053 | 0.030 |
| monoammonium glycyrrhizinate + | 30 | 0.086 | 0.032 |
| eucalyptus | | | |
| Sildenafil citrate, sucralose, dipotassium | 25 | 0.055 | 0.026 |
| glycyrrhizinate + eucalyptus | 30 | 0.095 | 0.033 |

1. A liquid pharmaceutical composition comprising at least 15 mg/ml of sildenafil citrate, between about 40% and about 60% aqueous ethanol, and having a pH of between about 5.5 and about 5.7.

2. (canceled)

3. The liquid pharmaceutical composition of claim 1, comprising at least 25 mg/ml of sildenafil citrate.

4. A liquid pharmaceutical composition comprising about 15 mg/ml of sildenafil citrate, between about 40% and about 60% aqueous ethanol, and having a pH of between about 5.5 and about 5.7.

5. (canceled)

6. The liquid pharmaceutical composition of claim 4, comprising at least 25 mg/ml of sildenafil citrate and having a pH of between about 5.5 and about 5.7.

7. The liquid pharmaceutical composition of claim 1, wherein the pH is about 5.6.

8. (canceled)

9. The liquid pharmaceutical composition of claim 1, comprising about 50% aqueous ethanol.

10. The liquid pharmaceutical composition of claim 1, further comprising sucralose.

11. A liquid pharmaceutical composition comprising at least 15 mg/ml of sildenafil citrate, about 50% aqueous ethanol, and having a pH of about 5.6.

12. (canceled)

13. The liquid pharmaceutical composition of claim 11, further comprising sucralose.

14. The liquid pharmaceutical composition of claim 13, further comprising a glycyrrhizinate.

15. The liquid pharmaceutical composition of claim 1, further compromising a flavoring agent other than sucralose.

16. The liquid pharmaceutical composition of claim 1, further compromising a bitterness blocking agent.

17. The liquid pharmaceutical composition of claim 1 which has shelf-life stability of at least 12 months.

18. (canceled)

19. The liquid pharmaceutical composition of claim 17 which has shelf-life stability of at least 36 months.

20. The liquid pharmaceutical composition of claim 1 which comprise less than 0.2% of impurity B at Day 1095 after initiation of stability testing.

21. An oral dosage form comprising the liquid pharmaceutical composition of claim 1, wherein said dosage form is a spray, solutions, drops, gelcaps, capsules or chewing gum.

22. (canceled)

23. A buccal, sublingual or inhaled dosage form comprising the liquid pharmaceutical composition of claim 1, wherein said dosage form is a thin-film, solution or spray.

24. (canceled)

25. A method of treating erectile dysfunction comprising administering, to a human subject in need thereof, the liquid pharmaceutical composition of claim 1.

26. The method of claim 25, wherein the volume of the liquid pharmaceutical composition administered to said human subject is less than 10 ml.

27-28. (canceled)

29. A method for the manufacture of the liquid pharmaceutical composition of claim 1 comprising adjusting the pH of the aqueous ethanol to between about 5.5 and about 5.7 and then mixing, at room temperature, said aqueous ethanol together with sildenafil citrate.

30-31. (canceled)
